(19) United States
(12) Patent Application Publication
(10) Pub. No.: US 2024/0075200 A1
(43) Pub. Date: Mar. 7, 2024

(54) NON-GRAVITATIONAL OCULAR DRUG DELIVERY DEVICE

(71) Applicant: TWENTY TWENTY THERAPEUTICS LLC, South San Francisco, CA (US)

(72) Inventor: Hojr Sedaghat Pisheh, South San Francisco, CA (US)

(21) Appl. No.: 18/241,512

(22) Filed: Sep. 1, 2023

Related U.S. Application Data
(60) Provisional application No. 63/403,041, filed on Sep. 1, 2022.

(51) Int. Cl. A61M 5/14

(52) U.S. Cl. CPC A61M 5/14; A61M 2202/0007; A61M 2210/0612

(57) ABSTRACT

A device for dispensing medicine to an eye from a container in a non-gravitational manner includes a housing defining a space for receiving the container of medicine. A pair of motors is provided on the housing on opposite sides of the receiving space. Each motor has an actuatable arm. A dispenser assembly is connected to the housing and includes a first end for securing to the container and a second end having a delivery port. The first and second ends are in fluid communication with each other and with the medicine in the container. The arms are actuatable into engagement with the container for urging the medicine out of the container, through the dispenser assembly, and out through the delivery port into the eye.

### RELATED APPLICATIONS

This application claims the benefit of U.S. Provisional Application Ser. No. 63/403,041, filed Sep. 1, 2022, the entirety of which is incorporated by reference herein.

### TECHNICAL FIELD

The present invention relates generally to drug delivery devices, and specifically to ocular drug delivery devices that force drug doses towards the eye.

### BACKGROUND

There are many reasons and conditions a patient may require medicated eye drops in liquid form. For instance, medicated drops can be used to treat Glaucoma, infection, eye irritation or even help combat dry eye through over-the-counter saline drops. Unfortunately, many people have significant difficulty holding objects near their eyes and/or are unable to properly deliver the drug to its target. This causes a reduction in user compliance, wasted drugs, and increased expense.

### SUMMARY

In one example, a device for dispensing medicine to an eye from a container in a non-gravitational manner includes a housing defining a space for receiving the container of medicine. A pair of motors is provided on the housing on opposite sides of the receiving space. Each motor has an actuatable arm. A dispenser assembly is connected to the housing and includes a first end for securing to the container and a second end having a delivery port. The first and second ends are in fluid communication with each other and with the medicine in the container. The arms are actuatable into engagement with the container for urging the medicine out of the container, through the dispenser assembly, and out through the delivery port into the eye.

In another example, a device for dispensing medicine into an eye from a container in a non-gravitational manner includes a housing defining a space for receiving a permanent magnet, an electromagnet, and a plunger. The electromagnet is fixed to the housing and the permanent magnet is longitudinally moveable with the plunger relative to the housing. The housing further includes a recess in fluid communication with the receiving space. A biasing member is positioned between the housing and the plunger for biasing the magnets away from one another. A syringe is provided in the recess and connected to the plunger. The syringe includes a flexible diaphragm and a reservoir for receiving medicine. The diaphragm is configured to deflect and urge the permanent magnet towards the electromagnet against the bias of the biasing member in response to pushing the container into the recess. The reservoir is configured to draw in medicine from the container in response to deflection of the diaphragm. The permanent magnet is urged away from the electromagnet in response to applying current to the electromagnet such that the plunger pushes the diaphragm to expel medicine from the reservoir into the eye.

In another example, a device for dispensing medicine to an eye from a container in a non-gravitational manner includes a housing defining a space for receiving the container of medicine. A pair of motors is provided on the housing on opposite sides of the receiving space. Each motor has an actuatable arm rotatable into engagement with the container. A reservoir is positioned downstream of the receiving space and in fluid communication with the container. A pump is positioned downstream of the reservoir and in fluid communication therewith. A nozzle is provided for delivering medicine from the pump to the eye.

In another example, a device for dispensing medicine to an eye in a non-gravitational manner includes a housing defining a receiving space and a nozzle. A piston is axially movable within the receiving space. Medicine is provided on a first side of the piston within the receiving space. A propellant material is provided on a second side of the piston opposite the first side. The piston is movable towards the medicine in response to expansion of the propellant material in order to expel the medicine out of the nozzle towards the eye.

Other objects and advantages and a fuller understanding of the invention will be had from the following detailed description and the accompanying drawings.

### BRIEF DESCRIPTION OF THE DRAWINGS

FIG. 1A is a schematic illustration of an example drug delivery device in accordance with the present invention.

FIG. 1B is a rear view of the device of FIG. 1A.

FIG. 2 is an exploded view of the device of FIG. 1A.

FIG. 3 is a schematic illustration of the device of FIG. 1A delivering medicine to an eye.

FIG. 4 is a schematic illustration of another example drug delivery device.

FIG. 5 is a schematic illustration of a micro syringe for use with the device of FIG. 4.

FIGS. 6A-6F illustrate operation of the device of FIG. 4.

FIG. 7 is a schematic illustration of another example drug delivery device.

FIG. 8 is a schematic illustration of another example drug delivery device.

### DETAILED DESCRIPTION

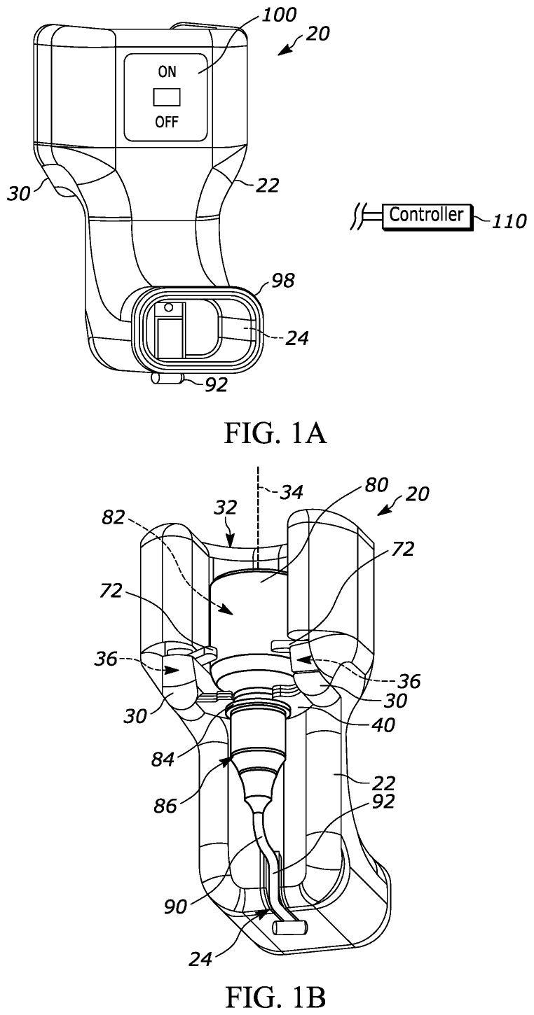

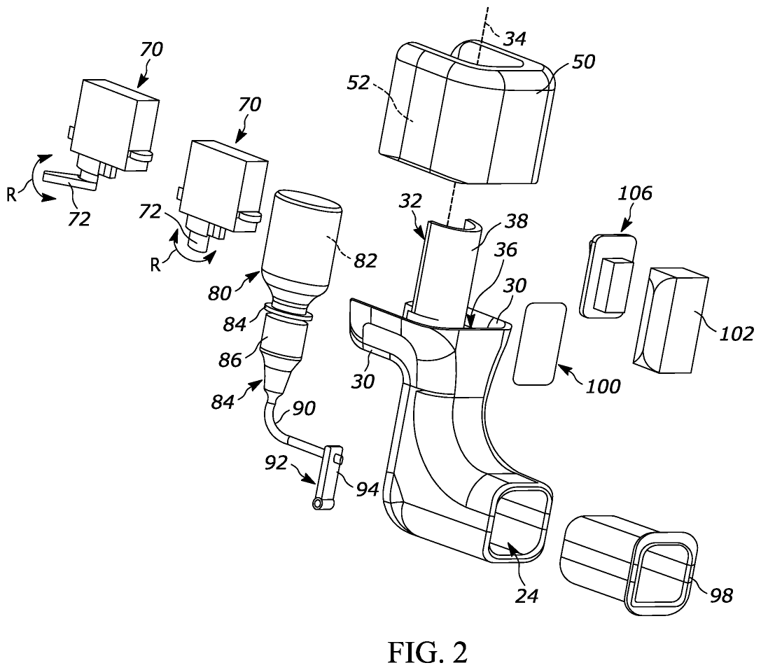

The present invention relates generally to drug delivery devices, and specifically to ocular drug delivery devices that force drug doses towards the eye. FIGS. 1A-2 illustrate a drug an example drug delivery device 20 in accordance with the present invention. The device 20 includes a housing 22 having, in one example, a generally z-shaped configuration. A passage 24 extends into a first side of the housing 22. An opening 26 extends into a second, opposite side of the housing 22 and connects to the passage 24. The passage 24 and the opening 26 are aligned with one another, e.g., co-extensive. A pair of first projections 30 extends from the second side of the housing 22. The projections 30 cooperate with the housing 22 to define a receiving space 32 extending longitudinally along a centerline 34.

A cavity 36 extends into both projections 30 and the housing 22. The cavity 36 extends partially around the receiving space 32 and can therefore have a u-shaped configuration. A mounting tab 38 extends from the housing 22 and upward (as shown) between the projections 30. A second projection 40 extends between the first projections 30 and into the receiving space 32. As shown, the second projection 40 is generally u-shaped.

A cap 50 having a u-shaped cavity 52 forms a snap-fit connection with the housing 22 and the mounting tab 38. A pair of openings 54 extends through the cap 50 on opposite sides of the cavity 52 and towards the centerline 34.

A pair of force applying members is provided in the cavities 36, 52. In one example, the force applying members include motors 70, e.g., servo motors. Each motor 70 has an arm 72 rotatable in the manner generally indicated at R. When positioned within the cavities 36, 52 the arms 72 are rotatable towards and away from the centerline 34 into and out of the receiving space 32.

A container 80 having medicine 82, e.g., eye medication such as Glaucoma medication or artificial tears, therein is provided in the receiving space 32. The container 80 includes a tapered adapter 84 configured to connect to the second projection 40. The container 80 can be a drug reservoir or commercially available eye dropper.

A dispenser assembly 86 extends from the adapter 84 and includes a tube 90 and another adapter 92. The adapter 92 includes an delivery port 94 that can be configured as a nozzle or atomizer. When the container 80 is provided on the housing 22, the dispenser assembly 86 extends through the receiving space 32 and away from the second projection 40. The adapter 92 is positioned in the opening 26 in the housing 22 such that the delivery port 94 extends into the passage 24. The adapter 92 can form a friction-fit with the opening 26 to helps maintain the position shown in FIG. 1B. A plug or cap 98 is inserted into the passage 24.

A battery 102 is provided in the cavity 52 and connected to the motors 70. The battery 102 can be rechargeable or non-rechargeable. A switch assembly 100 is also connected to the battery 102 and provided on the housing 22. The switch assembly 100 is actuated by the user/patient to enable or disable operation of the motors 70, as will be discussed.

A sensor assembly 106 is connected to the housing 22 so as to be located within the passage 24. In one example, the sensor assembly 106 includes an infrared light source, camera, and/or proximity sensor with eye tracking and blink detection software. A controller 110 can be connected to the motors 70, battery 102, switch assembly 100, and sensor assembly 110 for controlling operation and/or power distribution to the same.

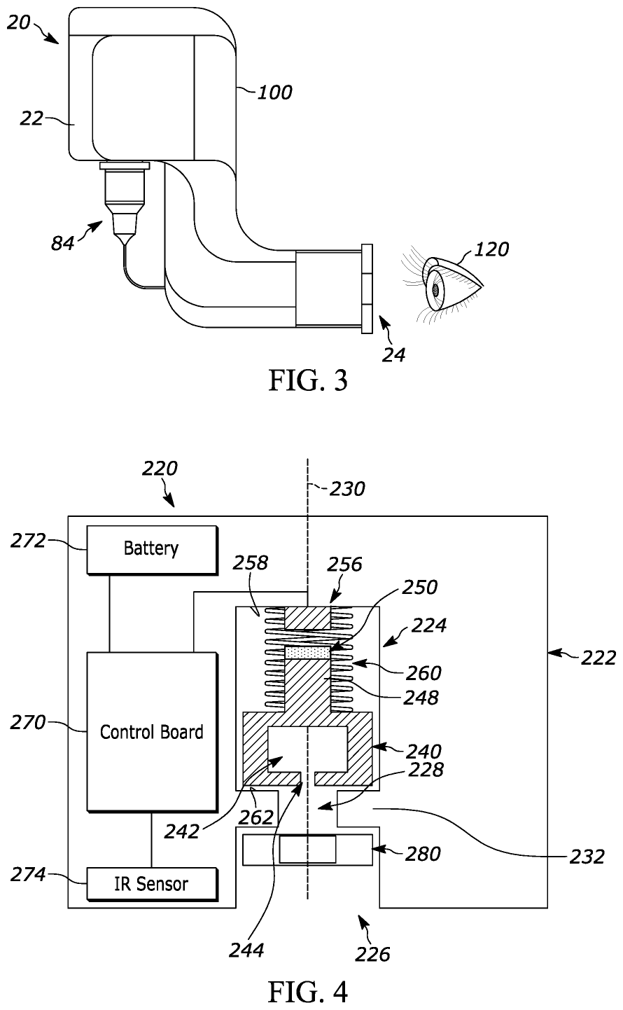

In operation, and referring to FIG. 3, when it is desirable to administer one or more drops of medicine 82 from the container 80, the user turns the switch assembly 100 to “ON”, and removes the cap 98 from the passage 24. The user then aligns the now unobstructed passage 24 with their eye 120. Advantageously, the device 20 allows the user to administer the medicine 82 without tipping their head back. In other words, the patient can look forward and simply align the passage 24 with the eye 120 within a plane that is generally horizontal/parallel with the ground.

With this in mind, once the passage 24 is aligned with the eye 120, the sensor assembly 106 monitors movement of the eye. More specifically, the sensor assembly 106 tracks blinking of the eye 120 and uses, for example, machine learning algorithms to anticipate when the user blinks. When the sensor assembly 106 determines a blink has occurred, the controller 110 immediately actuates the motors 70, thereby causing the arms 72 to rotate in the manner R towards one another and towards the container 80. The rotating arms 72 impact the container 80 and cause inward deformation thereof. This causes a predetermined amount/dosage of the medicine 82, e.g., a single dose, to be forced out of the container and into the dispenser assembly 86. The dose flows through the tube 90, into the adapter 92, and is expelled through the delivery port 94 into the passage 24 in the housing 20. Since the passage 24 is aligned with the eye 120, the dose exits the delivery port 94 aligned with the eye and therefore passes through the passage 24 directly into the open eye without the assistance of gravity.

It will be appreciated that the device 20 can be programmed to track, store, and send information related to dose dispensing to another device, e.g., computer or cellular phone. For instance, the controller 110 can be programmed to provide drug delivery alerts, logging data, and schedule future dosage alerts via notification, audio or haptic alert.

Another example device 220 for delivering medication to an eye 120 in a non-gravitational manner is illustrated in FIG. 4. The device 220 includes a housing 222 made of a plastic or metal. The housing 222 includes an interior chamber or space 224. A recess 226 formed in the exterior of the housing 222 extends towards the interior space 224. A shelf or radially extending wall 232 defines a passage 228 that connects the interior space 224 to the recess 226. The wall 232 also separates the interior space 224 from the recess 226. The interior space 226, recess 226, and passage 228 are all aligned a common centerline 230.

A plunger 240 is provided in the interior space 224. The plunger 240 is formed as a hollow body having an interior space 242. An opening 244 extends through the plunger 240 and into the interior space 242. A projection 248 extends longitudinally from plunger 240. The plunger 240, interior space 242, passage 244, and projection 248 are aligned all along the centerline 230. A permanent magnet 250 is secured to the end of the projection 248 opposite the plunger 240. In one example, the permanent magnet 250 has a soft, iron core.

An electromagnet 256 is secured to the housing 222 within the interior space 224 and aligned with the permanent magnet 250 along the centerline 230. A biasing member 260, such as a compression spring, encircles both magnets 250, 256 and the projection 248. The spring 260 abuts a first inner surface 258 of the housing 222 and extends into engagement with the plunger 240. The spring 260 is a compression spring that biases the piston 240 into engagement with a second inner surface 262 of the housing 222 opposing the first inner surface 258. In doing so, the spring 260 also biases the permanent magnet 250 into a first or initial position longitudinally spaced from the electromagnet 256.

A controller 270 is provided on the housing 222 and is connected to a battery 272, an infrared sensor 274, and the electromagnet 256. The controller 270 controls operation of and power distribution to the electromagnet 256 and the infrared sensor 274. The infrared sensor 274 is provided to detect proximity of the device 220 to the eye and/or detect eye blinking. A pressure ring 280 is provided in the recess 226 in the housing 222 and fixed in place relative thereto.

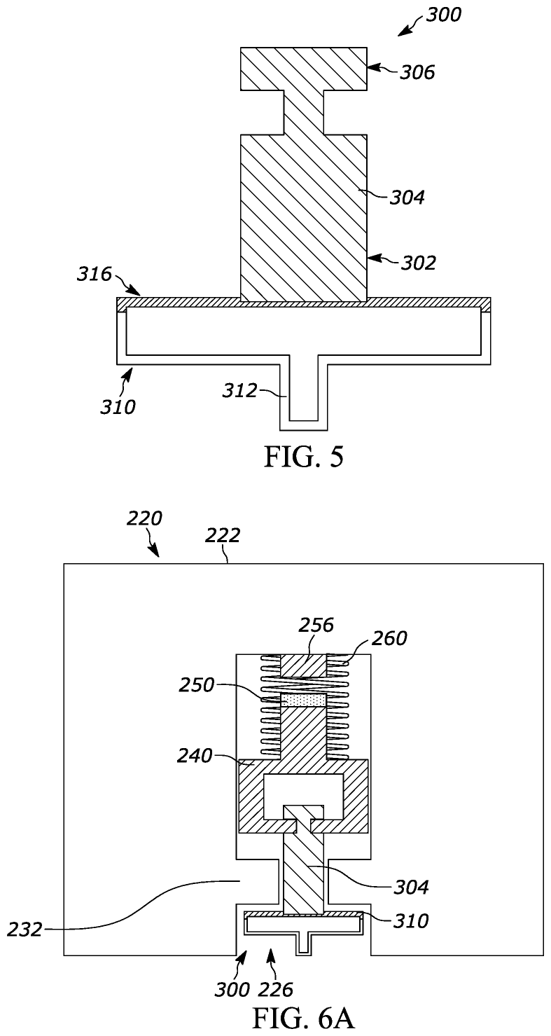

The device 220 is used in combination with a micro syringe 300 shown in FIG. 5. The syringe 300 includes a handle 302 having a body 304 and a projection or T-shaped member 306 extending therefrom. The handle 302 can be made of plastic, e.g., polyethylene terephthalate (PET). A flexible diaphragm 316 is provided on the end of the body 304 opposite the projection 306. The diaphragm 316 can be made of, for example, silicone rubber. A reservoir 310 is connected to the diaphragm 316 and includes a delivery port 312. The reservoir 310 has a fluid-tight connection with the diaphragm 316.

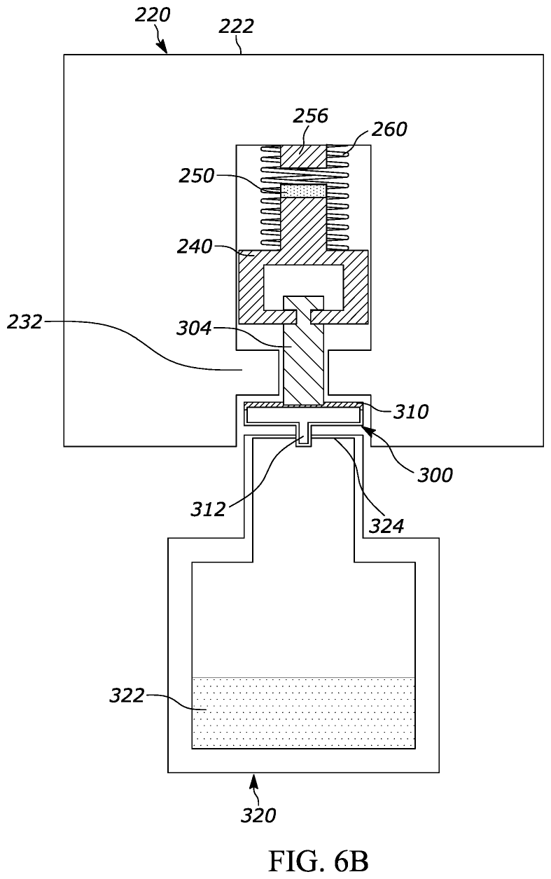

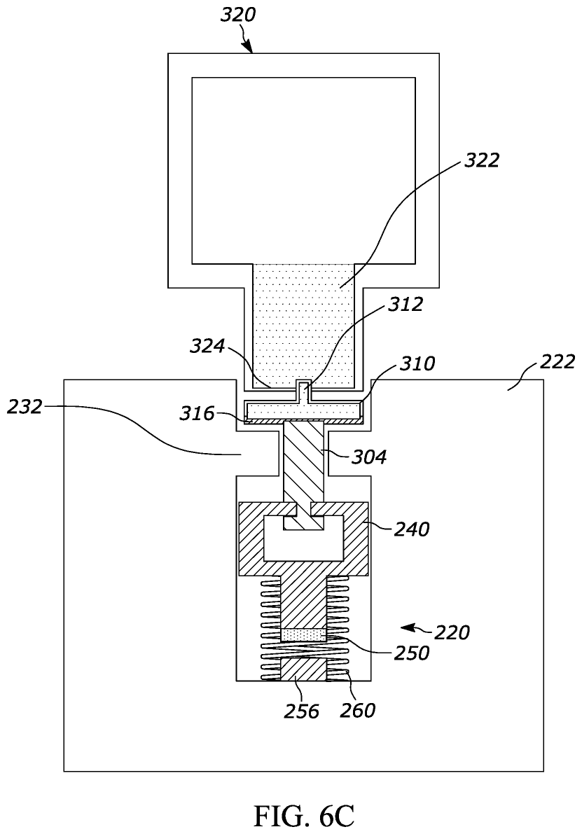

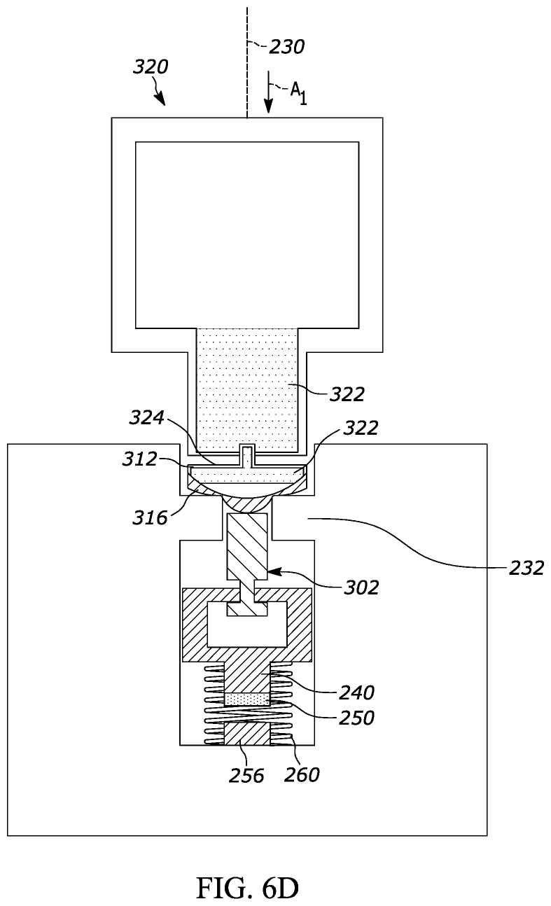

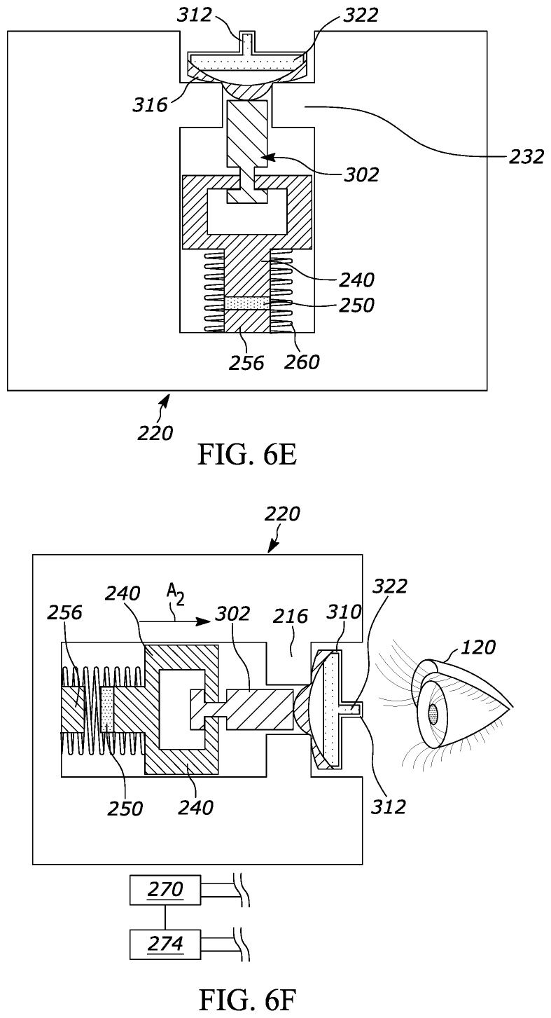

Operation of the device 220 with the syringe 300 is shown in FIGS. 6A-6F. In FIG. 6A, the handle 302 is inserted through the recesses 226, 228 in the housing 222, through the opening 244 in the piston 240, and into the interior space 242 thereof. This positions the diaphragm 316 and reservoir 310 within the recess 226 in the housing 222. This also secures the handle 302 within the interior space 242.

Referring to FIG. 6B, a container 320 is provided that stores liquid eye medicine 322. The container 320 is closed by a lid or septum 324. The container 320 is advanced towards the delivery port 312 of the reservoir 310 until the delivery port pierces the septum 324 of the container 320. This places the medicine 322 in fluid communication with the delivery port 312 and, thus, places the medicine in fluid communication with the reservoir 310. The device 220, syringe 300, and container 320 are then flipped or rotated as a collective unit such that the unit is upside down, i.e., the device resides below the syringe and container as shown in FIG. 6C. In this position, the bias of the spring 248 maintains the diaphragm 316 and reservoir 310 above and spaced from the shelf 232.

The user then pushes the container 320 along the centerline 230 towards the housing 222 in the direction A₁ in FIG. 6D and against the bias of the spring 248. Pushing the container 320 forces the reservoir 310 into the pressure ring 280, which holds the reservoir in place within the recess 226. At the same time, pushing the container 320 in the direction A₁ forces the medicine 322 out of the container through the delivery port 312 and into the reservoir 310. This expands the reservoir 310, thereby causing the diaphragm 316 connected thereto to deflect through the passage 228. More specifically, the diaphragm 316 deflects such that the generally central portion thereof passes into the passage 228 while the outer/peripheral portion deflects away from the wall 232. In other words, the diaphragm 316 transitions from a generally planar shape to a generally concave shape.

Transitioning the diaphragm 316 creates a vacuum in the reservoir 310 which, in turn, help draw medicine 322 from the container 320, through the delivery port 312, and into the reservoir. The diaphragm 316 and/or delivery port 312 can be configured to draw a predetermined amount of the medicine 322 into the reservoir 310, e.g., a single dose.

The deflecting diaphragm 316 pushes the handle 302—and thereby pushes the plunger 240 secured thereto—against the bias of the spring 260 in the direction A₁ until the magnets 250, 256 engage one another. This relationship is maintained by a combination of the pressure ring 280 holding the reservoir 320 in place and the fluid pressure within the reservoir. That said, once the medicine 322 stops flowing into the reservoir 310 the container 320 is removed from the device 220 (FIG. 6E).

Referring to FIG. 6F, the device 220 is now positioned such that the delivery port 312 of the reservoir 310 is aligned with and pointed at the eye 120. The infrared sensor 274 helps the device 220 identify when the delivery port 312 is positioned the desired distance from the eye 120. To this end, the infrared sensor 274 continuously sends signals to the controller 270 indicative of the spacing. Once a desired spacing occurs, the controller 270 automatically sends power to the electromagnet 256, which creates a repelling force between the magnets 250, 256. This causes the permanent magnet 250 to be urged in the direction A₂ and, thus, the plunger 240 and handle 302 connected thereto are urged in the direction A₂. Consequently, the spring 260 lengthens to help push the plunger 240 and handle 302 towards the eye 120 in the direction A₂.

Since the reservoir 310 is fixed in position on the housing 222, the moving handle 302 pushes against the concave diaphragm 316 in the direction A₂, which squeezes or collapses the reservoir 310. The collapsing reservoir 310 pressurizes the medicine 322 therein. As a result, the medicine 322 is forced out of the delivery port 312 and expelled into the eye 120. The controller 270 then cuts the power supply to the electromagnet 256. It will be appreciated that the same blink detection, eye tracking, and data storing/tracking features mentioned above regarding the device 20 can likewise be implemented into the device 220.

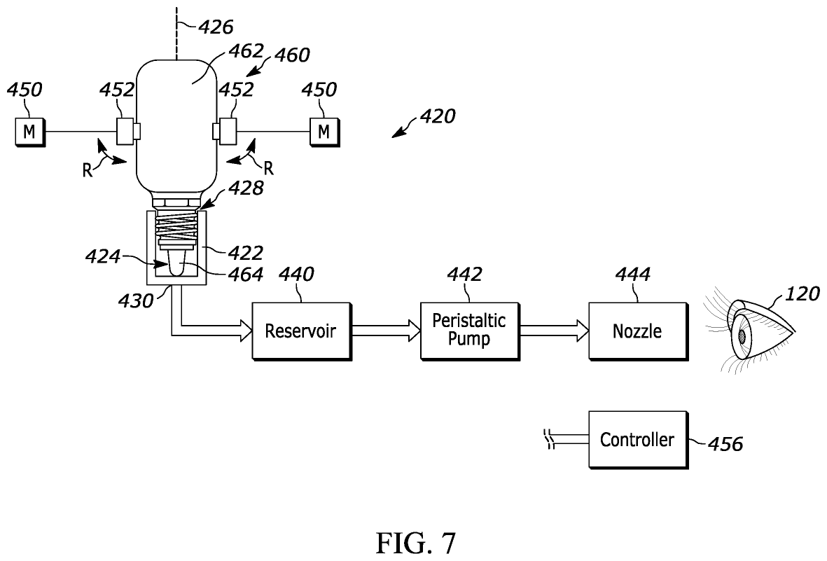

Another example device 420 for delivering medication to an eye 220 in a non-gravitational manner is illustrated in FIG. 7. The device 420 includes a housing 422 made of a plastic or metal. The housing 422 includes an interior chamber or space 424 extending longitudinally along a centerline 426. An opening 428 extends through one end of the housing 422 to the interior space 424. An opening 430 extends through the other end of the housing 422 to the interior space 424.

A reservoir 440 is fluidly connected to the opening 430. A pump, such as a peristaltic pump 442, is fluidly connected to the reservoir at a downstream side thereof. An atomizing nozzle 444 is fluidly connected to the pump 442 at a downstream end thereof. Alternatively, an air pump (not shown) can be provided on the downstream side of the pump 442 and fluidly connected thereto.

A pair of force applying members, e.g., servo motors 450, is connected to the housing 422 on opposite sides of the centerline 426. Each motor 450 includes an arm 452 rotatable towards and away from the centerline 426 in the manner generally indicated at R. A controller 456 is connected to the pump 442 and motors 450 for controlling operation and/or power distribution to the pump and motors.

A container 460 having medicine 462, e.g., eye medication such as Glaucoma medication or artificial tears, therein is provided in the receiving space 424 and between the arms 452 of the motors 450. The container 460 includes a delivery port 464. The container 460 can be a drug reservoir or commercially available eye dropper. It will be appreciated that the same blink detection, eye tracking, and data storing/tracking features mentioned above regarding the device 20 can likewise be implemented into the device 420.

In operation, once the nozzle 444 is aligned with the eye 120, the controller 452 actuates the motors 450 and the pump 442. The arms 452 of the motors 450 rotate in the manner R towards one another and towards the container 460. The rotating arms 452 impact the container 460 and cause a predetermined amount, e.g., a single dose, of the medicine 462 to be forced out of the container and into the reservoir 440. The dose flows through the reservoir 440, into the pump 442, and is expelled through the nozzle 444 aligned with the eye 120. Consequently, the dose passes through the nozzle 444 and is sprayed/expelled directly into the open eye 120.

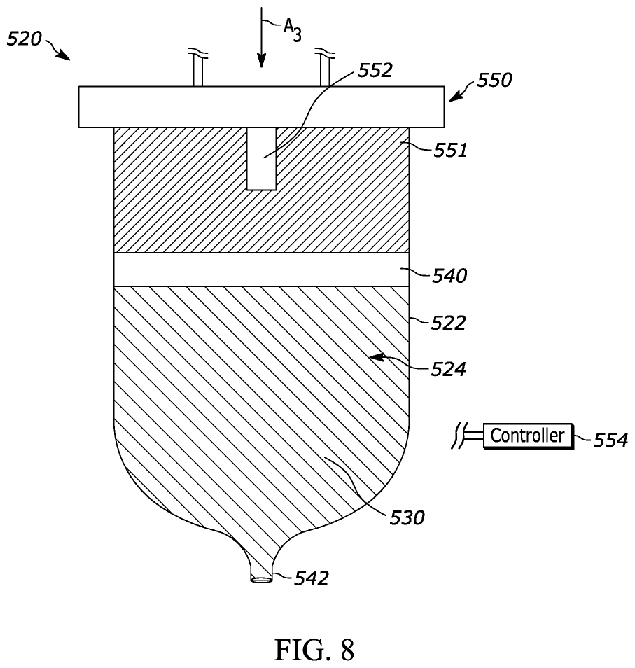

In another example shown in FIG. 8, the medicine can non-gravitationally delivered to the eye by a thermodynamic driver, such as by igniting a propellant. In this construction the drug delivery device 520 includes a housing 522 defining a receiving space 524 for medicine 530. A piston 540 is provided in the receiving space 524 and axially movable therein. A nozzle 542 provided on the end of the housing 522 helps direct the medicine 530 towards the eye.

A thermodynamic driver 550 is provided on the housing 522. In one example, the thermodynamic driver 550 includes a propellant material 551, such as nitrocellulose, provided in the receiving space 524 on a side of the piston 540 opposite the medicine 530. As shown, the medicine 530 is positioned downstream or in front of the piston 540 while the propellant material 551 is positioned upstream or behind the piston. An igniter 552 extends into the receiving space 524 and into proximity/contact with the propellant material 551. The igniter 552 is connected to a controller 554 for selectively actuating the same.

In operation, the nozzle 542 is oriented towards the eye and the user either presses an actuating button (not shown) to actuate the igniter 552 or the controller 554 automatically actuates the igniter based on blink detection. Regardless, current is passed through a resistor in the igniter 552 until a predetermined temperature thereof is reached.

When this occurs, the propellant material 551 is ignited to produce pressurized gases that expand. These gases act on the piston 540 to move the piston axially within the housing 522 towards the nozzle 542 in the manner A₃. As a result, the piston 540 pushes the medicine 530 out of the device 520 through the nozzle 542 towards the eye. It will be appreciated that other methods of generating the pressurized gas from the propellant material 551, such as by electrical heating or inductive power transfer, are contemplated.

In any case, in one example the device 520 can expel about 50 μL of medicine 530 through a 1 mm diameter nozzle 542 at a rate of about 3 m/s. The nozzle 542 can be configured to provide a desired spray pattern of the medicine 530 towards the eye. In one instance, the device 520 can only be used for individual doses before being discarded.

This configuration is advantageous in that the user never directly handles the medicine or is responsible for administering the proper dosage as the housing is already pre-filled with correct amount. Furthermore, the unidose configuration of the device alleviates the need for using medications with preservatives. Moreover, it will be appreciated that the piston and propellant material are hermetically sealed within the housing to promote efficient ignition of the material and driving of the piston. It will also be appreciated that the thermodynamic driver can be implemented into other devices shown and described herein in order to drive, for example, the peristaltic pump or other pistons shown and described.

The aforementioned drug delivery devices are advantageous in that existing medicine containers made by pharmaceutical companies do not need alteration in order to be used with the device. Furthermore, using non-gravitational means to expel the drug into the eye allows the user to deliver the drug in a position-agnostic manner. This is convenient for patients who are not able to align the eye dropper with the eye and/or have difficulty leaning backwards to ensure gravitational ocular drug delivery. With that said, the drug delivery using non-gravitational means is more precise and efficient, thereby reducing waste.

It will be appreciated that the drug delivery devices shown and described herein can be modified to include, for example, a speaker, dose indicator, securing strap, charger/holder, and/or carrying case.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A device for dispensing medicine to an eye from a container in a non-gravitational manner, comprising:
a housing defining a space for receiving the container of medicine;
a pair of motors provided on the housing on opposite sides of the receiving space, each motor having an actuatable arm; and
a dispenser assembly connected to the housing and having a first end for securing to the container and a second end having a delivery port, the first and second ends being in fluid communication with each other and with the medicine in the container;
wherein the arms are actuatable into engagement with the container for urging the medicine out of the container, through the dispenser assembly, and out through the delivery port into the eye.

2. The device according to claim 1, wherein the housing further includes an outlet passage in fluid communication with the delivery port of the dispenser assembly.

3. The device according to claim 1, wherein the arms are automatically brought into engagement with the container in response to detecting a blink of the eye.

4. The device according to claim 1, wherein a projection extends from the housing into the receiving space for forming a snap-fit connection with the container.

5. The device according to claim 1, wherein the housing has a generally z-shaped configuration.

6. The device according to claim 1, further comprising a switch assembly actuatable by a user to selectively enable or disable actuation of the motors.

7. A device for dispensing medicine into an eye from a container in a non-gravitational manner, comprising:
a housing defining a space for receiving a permanent magnet, an electromagnet, and a plunger, wherein the electromagnet is fixed to the housing and the permanent magnet is longitudinally moveable with the plunger relative to the housing, the housing further includes a recess in fluid communication with the receiving space;
a biasing member positioned between the housing and the plunger for biasing the magnets away from one another; and
a syringe provided in the recess and connected to the plunger, the syringe including a flexible diaphragm and a reservoir for receiving medicine, wherein the diaphragm is configured to deflect and urge the permanent magnet towards the electromagnet against the bias of the biasing member in response to pushing the container into the recess, the reservoir being configured to draw in medicine from the container in response to deflection of the diaphragm, wherein the permanent magnet is urged away from the electromagnet in response to applying current to the electromagnet such that the plunger pushes the diaphragm to expel medicine from the reservoir into the eye.

8. The device according to claim 7, wherein the biasing member comprises a spring that encircles the magnets as well as a projection extending from the plunger.

9. The device according to claim 7, wherein the biasing member helps push the plunger towards the diaphragm.

10. The device according to claim 7, wherein the diaphragm is deflected from a planar shape to a concave shape in response to pushing the container into the recess.

11. The device according to claim 7, further comprising a sensor for detecting at least one of an orientation and a blink of the eye.

12. The device according to claim 7, wherein the syringe further comprises a handle connected to the diaphragm and having a projection extending through the recess in the housing into the receiving space such that the handle pushes the plunger towards the electromagnet in response to deflection of the diaphragm.

13. A device for dispensing medicine to an eye from a container in a non-gravitational manner, comprising:
a housing defining a space for receiving the container of medicine;
a pair of motors provided on the housing on opposite sides of the receiving space, each motor having an actuatable arm rotatable into engagement with the container;
a reservoir positioned downstream of the receiving space and in fluid communication with the container;
a pump positioned downstream of the reservoir and in fluid communication therewith; and
a nozzle for delivering medicine from the pump to the eye.

14. The device according to claim 13, wherein the pump comprises a peristaltic pump.

15. The device according to claim 13, wherein the nozzle is an atomizing nozzle.

16. A device for dispensing medicine to an eye in a non-gravitational manner, comprising:
a housing defining a receiving space and a nozzle;
a piston axially movable within the receiving space;
medicine provided on a first side of the piston within the receiving space; and
a propellant material provided on a second side of the piston opposite the first side, wherein the piston is movable towards the medicine in response to expansion of the propellant material in order to expel the medicine out of the nozzle towards the eye.

17. The device according to claim 16, wherein the propellant material comprises nitrocellulose.

18. The device according to claim 16, further comprising an igniter for igniting the propellant material.

19. The device according to claim 16, wherein the propellant material is electrically heated to cause expansion thereof.
